SEQUENCE LISTING (20 sequences)

SEQ ID NO: 1 - DNA, 1272 bp, synthetic construct
  source 1..1272: mol_type = other DNA; note = Soybean GmPP2C11 gene
atgcctggcattctctcaaggctgaatgcaaccatttactgtcgcattcgggaagcaataacaaggcaacaaggagtgccgacatccttgtacagaagttcagctttgcctctttgttcgctcacttcaagtcacacacttcacacaaaatcaatgattactgcctcacattccaatgctatgctgggagatgtttatgcttatggcttaatctcaggtcgtggtagtgtgcgagacttcacaaagcctgctgtaggttgcttgaggggtagtgtgaatctaaggagactacaaccattgtatggtcctttgagttttgggtgttctacttttgatgctaataggaggatccgggattcgagtttgctgcatggatcatggctcaagaatttctcagcctcttcttctgcttgctactcagccggggctgcacatgctgtctcatttgatggaagccctcctgatgaacagcttgcaaattcctctttttcgcctgacccaactattgttggtggtaagcccttgaagatgttgtcaggatcttgttacctaccacatccagataaggaggagacaggaggagaggatgctcattttatttgcacggatgaacaagcaattggtgttgctgatggtgtaggtggctgggcggatgttggcgttaatgcaggactgtttgcacaggaacttatatccaatttagtgagagcaattcaaaaggagcctaaaggttcttttaatctgacaagagtgttaagggaagctcacgcaaatacaaaggtcaagggttcatcaacagcttgtattgttgctcttactgacaagggactacatgctattaatttgggtgacagtggattcattgtggttagagatggatgcaccattttcgagtccccttctcaacagcatgacttcaatttcccatatcagttggagagtgggaatggcgctgatctacccagctctggtgaggtttttaccatacccgttgcttcaggagatgtggtcatttctggaacagatggtttatttgacaatttgtacaacagtgagatcactggagttgttgaacatgcaattagggctggactagagccccaagtaactgctcagaagatagcagcactggctcgtcagcgagcactgtccaagagtagtcggacaccattttctactgctgctgaaaaagctggattctgttattatggtggcaaacttgatgacataactgttgttgtctcatacatatcaggttcagtgagtgagtga

SEQ ID NO: 2 - protein, 423 aa, synthetic construct
  source 1..423: note = GmPP2C11 protein
MPGILSRLNATIYCRIREAITRQQGVPTSLYRSSALPLCSLTSSHTLHTKSMITASHSNAMLGDVYAYGLISGRGSVRDFTKPAVGCLRGSVNLRRLQPLYGPLSFGCSTFDANRRIRDSSLLHGSWLKNFSASSSACYSAGAAHAVSFDGSPPDEQLANSSFSPDPTIVGGKPLKMLSGSCYLPHPDKEETGGEDAHFICTDEQAIGVADGVGGWADVGVNAGLFAQELISNLVRAIQKEPKGSFNLTRVLREAHANTKVKGSSTACIVALTDKGLHAINLGDSGFIVVRDGCTIFESPSQQHDFNFPYQLESGNGADLPSSGEVFTIPVASGDVVISGTDGLFDNLYNSEITGVVEHAIRAGLEPQVTAQKIAALARQRALSKSSRTPFSTAAEKAGFCYYGGKLDDITVVVSYISGSVSE

SEQ ID NO: 3 - RNA, 43 nt, synthetic construct
  source 1..43: mol_type = other RNA; note = sgRNA GmPP2C11-sgF1
taatctgacaagagtgttaagttttagagctagaaatagcaag

SEQ ID NO: 4 - RNA, 42 nt, synthetic construct
  source 1..42: mol_type = other RNA; note = sgRNA GmPP2C11-sgR1
ttaacactcttgtcagattaaatccatatgttttcctgggac

SEQ ID NO: 5 - RNA, 43 nt, synthetic construct
  source 1..43: mol_type = other RNA; note = sgRNA GmPP2C11-sgF2
tcccatatcagttggagagtgttttagagctagaaatagcaag

SEQ ID NO: 6 - RNA, 43 nt, synthetic construct
  source 1..43: mol_type = other RNA; note = sgRNA GmPP2C11-sgR2
actctccaactgatatgggatgaccagacatgtcacgcttagt

SEQ ID NO: 7 - RNA, 43 nt, synthetic construct
  source 1..43: mol_type = other RNA; note = sgRNA GmPP2C11-sgF3
gttgaacatgcaattagggcgttttagagctagaaatagcaag

SEQ ID NO: 8 - RNA, 42 nt, synthetic construct
  source 1..42: mol_type = other RNA; note = sgRNA GmPP2C11-sgR3
gccctaattgcatgttcaacaatccatatgttttcctgggac

SEQ ID NO: 9 - DNA, 20 bp, synthetic construct
  source 1..20: mol_type = other DNA; note = Upstream primer GmPP2C11-F
acacccgtggtttcacatta

SEQ ID NO: 10 - DNA, 22 bp, synthetic construct
  source 1..22: mol_type = other DNA; note = Downstream primer GmPP2C11-R
tctcttccaccagttcaaacat

SEQ ID NO: 11 - DNA, 19 bp, synthetic construct
  source 1..19: mol_type = other DNA; note = Tubulin forward primer
ggagttcacagaggcagag

SEQ ID NO: 12 - DNA, 20 bp, synthetic construct
  source 1..20: mol_type = other DNA; note = Tubulin reverse primer
cacttacgcatcacatagca

SEQ ID NO: 13 - DNA, 20 bp, synthetic construct
  source 1..20: mol_type = other DNA; note = primer GmPP2C11-qPCR-F
tgctgatggtgtaggtggct

SEQ ID NO: 14 - DNA, 20 bp, synthetic construct
  source 1..20: mol_type = other DNA; note = Primer GmPP2C11-qPCR-R
atttgcgtgagcttccctta

SEQ ID NO: 15 - DNA, 40 bp, synthetic construct
  source 1..40: mol_type = other DNA; note = Primer GmPP2C11-F
acaaatctatctctctcgagatgcctggcattctctcaag

SEQ ID NO: 16 - DNA, 37 bp, synthetic construct
  source 1..37: mol_type = other DNA; note = Primer GmPP2C11-R
gctcaccatggatccctcactcactgaacctgatatg

SEQ ID NO: 17 - DNA, 21 bp, synthetic construct
  source 1..21: mol_type = other DNA; note = Primer Bar-F
cgagacaagcacggtcaactt

SEQ ID NO: 18 - DNA, 21 bp, synthetic construct
  source 1..21: mol_type = other DNA; note = Primer Bar-R
aaacccacgtcatgccagttc

SEQ ID NO: 19 - DNA, 20 bp, synthetic construct
  source 1..20: mol_type = other DNA; note = Primer GmPP2C11-qPCR-F
tgctgatggtgtaggtggct

SEQ ID NO: 20 - DNA, 20 bp, synthetic construct
  source 1..20: mol_type = other DNA; note = Primer GmPP2C11-qPCR-R
atttgcgtgagcttccctta

(19) United States
(12) Patent Application Publication
(10) Pub. No.: US 2026/0176647 A1
(43) Pub. Date: Jun. 25, 2026

(54) USE OF SOYBEAN PROTEIN PHOSPHATASE FAMILY GENE GmPP2C11

(71) Applicant: NANJING AGRICULTURAL UNIVERSITY, Nanjing (CN)

(72) Inventors: Fang HUANG, Nanjing (CN); Deyue YU, Nanjing (CN); Zhongyi YANG, Nanjing (CN); Dezhou HU, Nanjing (CN); Guizhen KAN, Nanjing (CN); Fei WU, Nanjing (CN); Xuan CUI, Nanjing (CN)

(21) Appl. No.: 18/847,612

(22) PCT Filed: Dec. 27, 2023
(86) PCT No.: PCT/CN2023/142175

§ 371 Date: Sep. 16, 2024

(30) Foreign Application Priority Data
Jun. 13, 2023 (CN) 202310697372.3

(51) Int. Cl. C12N 15/82; C12N 9/22; C12N 15/11

(52) U.S. Cl. CPC C12N 15/8262; C12N 9/226; C12N 15/11; C12N 15/8205; C12N 15/8213; C12Y 301/03016; C12N 2310/20

(57) ABSTRACT

Use of a soybean protein phosphatase family gene GmPP2C11 is provided. A soybean gene GmPP2C11 encoding a GmPP2C11 protein has the nucleotide sequence of SEQ ID NO: 1. A PSCM-GmPP2C11 knockout vector is genetically transformed into soybean, and it is found that a resulting gene-edited material shows significantly higher hundred-seed weight and yield per plant than those of a receptor Jack. This indicates that the soybean gene GmPP2C11 encoding the soybean GmPP2C11 protein may increase the seed weight and yield of a plant through genetic engineering, thereby providing technical services for increasing the yield of crops, especially soybean.

### CROSS REFERENCE TO RELATED APPLICATION

The present application is a national stage application of PCT application No. PCT/CN2023/142175 filed on Dec. 27, 2023, which claims priority to a Chinese Patent Application CN202310697372.3 filed to the China National Intellectual Property Administration (CNIPA) on Jun. 13, 2023 and entitled “USE OF SOYBEAN PROTEIN PHOSPHATASE FAMILY GENE GmPP2C11”, both of which are incorporated herein by reference in its entirety.

### REFERENCE TO SEQUENCE LISTING

A computer readable XML file entitled “GWPCTP20240604451_seqlist”, that was created on Aug. 28, 2024, with a file size of about 23,980 bytes, contains the sequence listing for this application, has been filed with this application, and is hereby incorporated by reference in its entirety.

### TECHNICAL FIELD

The present disclosure belongs to the technical field of plant genetic engineering, and relates to use of a soybean protein phosphatase family gene GmPP2C11.

### BACKGROUND

Soybean [Glycine max (L.) Merr.] is an important food and oil crop rich in protein and oil, and also serves as a main source of protein in animal feed. China is a country of origin of the soybean, but the current soybean production in China is far from meeting people's growing demand, such that increasing domestic soybean production is an urgent issue to be addressed. The research on soybean yield traits is particularly important.

In a study of our lab, a quantitative trait locus (QTL) qSW11 on chromosome 11 that is tightly linked to soybean hundred-seed weight was identified through linkage analysis and genome-wide association study (GWAS) of the soybean yield traits, and then a candidate gene GmPP2C11 (Glyma.11g065900V2.0) was selected from the QTL. This gene encodes a soybean protein phosphatase 2C (PP2C), which may be involved in the reversible phosphorylation of proteins, and plays an important role in the signal transduction of soybean growth, development, and resistance. Therefore, the biological function of the GmPP2C11 is further explored.

GmPP2C11 belongs to the PP2C family. Currently, a total of 134 PP2Cs have been identified in soybean (Fan et al., 2020). An important feature of plant PP2Cs is that there are 11 conserved structural sub-regions in their catalytic region (Bork et al., 1996). PP2Cs are monomeric enzymes that lack regulatory subunits compared to pentose phosphate pathway (PPP) protein phosphatases, and the activity of PP2C depends on the regulation by magnesium or manganese ions. Other ions such as copper and zinc ions can inactivate PP2C by competing with PP2C-dependent ions (Lumpp et al., 1998; Baudouin et al., 1999). Most PP2Cs in plant have a conserved catalytic region at their C-terminal. Meanwhile, their N-terminal is structurally diverse, showing various lengths of extended regions. These regions, such as transmembrane regions, often contain sequences related to intracellular signal transduction, giving different PP2Cs different functions. For example, the N-terminal of PP2C DBPl in tobacco has a transcription factor sequence that can bind to the promoter of defense-related genes (Carrasco et al., 2003). Truncation on the N-terminal extended region of the ABIl gene results in an increased protein phosphatase activity (Sheen, 1998).

PP2Cs are involved in important plant life processes, such as growth and development, biotic stress, and abiotic stress of plants. Under drought stress, core ABA signaling components CsPYL1, CsPYL2, CsPP2C2, and CsSnKR2.2 are found to be up-regulated in roots, stems, and leaves of cucumber seedlings, among which the CsPP2C2 is a type 2C protein phosphatase (Wang et al., 2012). The MP2C in alfalfa is involved in the response of alfalfa to drought stress by regulating mitogen-activated protein kinase (MAPK) activity (Meskiene et al., 2003). Screening of Arabidopsis thaliana mutants for abscisic acid (ABA) sensitivity identified AtPP2CA as a negative regulator of ABA signaling in Arabidopsis thaliana (Kuhn et al., 2006).

### SUMMARY

An objective of the present disclosure is to disclose use of a soybean PP2C family gene GmPP2C11.

The objective of the present disclosure can be achieved by the following technical solutions:

The present disclosure provides use of a soybean gene GmPP2C11 encoding a GmPP2C11 protein in regulation of a thousand-seed weight of Arabidopsis thaliana and a hundred-seed weight and a yield per plant of soybean by genetic engineering; where the soybean gene GmPP2C11 encoding the GmPP2C11 protein has the nucleotide sequence of SEQ ID NO: 1.

In some embodiments, a plant height, the hundred-seed weight, and the yield per plant of soybean is increased by inhibiting expression of the soybean gene GmPP2C11 encoding the GmPP2C11 protein.

In some embodiments, the plant height, the hundred-seed weight, and the yield per plant of the soybean is increased by knocking out the soybean GmPP2C11 gene.

The present disclosure further provides a recombinant expression vector including the soybean gene GmPP2C11 encoding the GmPP2C11 protein in regulation of a thousand-grain weight of Arabidopsis thaliana by genetic engineering; where the soybean gene GmPP2C11 encoding the GmPP2C11 protein has the nucleotide sequence of SEQ ID NO: 1.

The present disclosure further provides use of a single guide RNA (sgRNA) knockout vector including the soybean gene GmPP2C11 encoding the GmPP2C11 protein in improvement of a plant height, a hundred-seed weight, and a yield per plant of soybean by genetic engineering; where the soybean gene GmPP2C11 encoding the GmPP2C11 protein has the nucleotide sequence of SEQ ID NO: 1.

When GmPP2C11 of the present disclosure is used to construct a plant expression vector, any enhanced promoter or inducible promoter can be added before a transcription initiation nucleotide. In order to facilitate identification and screening of a transgenic plant cell or plant, the plant expression vector used can be processed, for example, by adding selectable marker genes (a GUS gene, a GFP gene and the like) expressed in plants or antibiotic resistance markers (gentamicin resistance marker, kanamycin resistance marker, and hygromycin resistance marker and the like). Considering safety of transgenic plants, it is also possible to directly screen transformed plants by phenotypic traits without adding any selectable marker genes.

The plant expression vector carrying the GmPP2C11 of the present disclosure can be transformed into plant cells or tissues by conventional biological methods, such as Ti plasmid, Ri plasmid, plant viral vector, direct DNA transformation, microinjection, electroconduction, and Agrobacterium-mediated method, and then obtained transformed plant tissues are cultured into plants. The transformed plant host can be a monocotyledon such as rice, wheat, and corn, or a dicotyledon such as tobacco, Arabidopsis thaliana, soybean, rapeseed, cucumber, tomato, poplar, turf grass, and alfalfa.

### Beneficial Effects:

In the present disclosure, GmPP2C11 belongs to the PP2C family. Tissue expression analysis has revealed that GmPP2C11 is mainly expressed at a higher level in pods, and subcellular localization has showed that the GmPP2C11 protein is mainly localized in chloroplasts. GmPP2C11 is knocked out using a gene editing vector PSCM-GmPP2C11, so as to regulate the plant height, hundred-seed weight, and yield per plant of soybean. A GmPP2C11 gene-edited material has significantly improved plant height, hundred-seed weight, and yield per plant of the soybean compared to a control Jack. The present disclosure further provides use of the gene in regulating seed development and yield of plants. The yield of crops may be increased by targeted modification of their seed size.

### BRIEF DESCRIPTION OF THE DRAWINGS

The present disclosure is described in more detail with reference to the accompanying drawings and examples. To describe the technical solutions in the examples of the present disclosure or in the prior art more clearly, the accompanying drawings required for the examples will be briefly described below.

FIG. 1 shows cloning of the GmPP2C11 gene; where primers are designed based on GmPP2C11 sequence information predicted by the phytozome website, and PCR amplification is conducted using leaf cDNA of Nannong 1138-2 as a template to obtain a 1,272 bp DNA fragment; after sequencing analysis, sequence information of the fragment is consistent with a sequence predicted by the phytozome website, that is, the 1,272 bp DNA fragment is the GmPP2C11 gene; marker is 2 k, 100 bp, 250 bp, 500 bp, 750 bp, 1,000 bp, and 2,000 bp;

FIG. 2 shows tissue expression analysis of the GmPP2C11 gene; where real-time fluorescence quantitative polymerase chain reaction (PCR) is conducted to study the expression of GmPP2C11 in different soybean tissues of Nannong 1138-2 and Kefeng 1, and the different soybean tissues are roots, stems, leaves, flowers, pods, and seeds;

FIG. 3 shows subcellular localization of the GmPP2C11 gene;

FIG. 4 shows a bar test strip detection of a GmPP2C11 gene-edited soybean plant, where 1 represents a receptor material; 2 to 6 represent 5 To generation transgenic lines, respectively;

FIG. 5 shows positive identification of the GmPP2C11 gene-edited soybean plant; where the markers represent 2 k, 100 bp, 250 bp, 500 bp, 750 bp, 1,000 bp, and 2,000 bp; WT represents an amplification result of the receptor material Jack; 1 and 7 represent positive controls, respectively; 2 to 6 represent 5 transgenic plants KO-1, KO-3, KO-5, KO-6, and KO-7 detected in the T₀ generation, respectively;

FIG. 6 shows expression analysis of the GmPP2C11 gene in the gene editing material and the receptor material Jack;

FIGS. 7A-7B show a comparison of plant height at maturity of the GmPP2C11 gene-edited material and the receptor Jack of soybean; and

FIGS. 8A-8C show a comparison of hundred-seed weight (b) and yield per plant (c) of the GmPP2C11 gene-edited material and the receptor Jack of soybean.

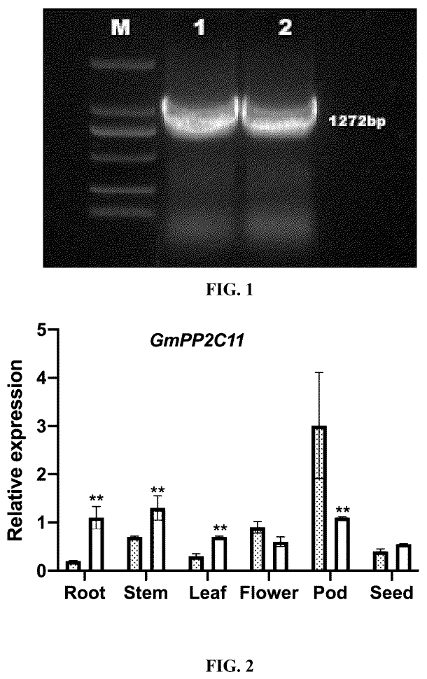

### DETAILED DESCRIPTION OF THE EMBODIMENTS

The present disclosure is further described in detail below in conjunction with specific examples and with reference to data.

### Example 1

### Cloning and Identification of Soybean GmPP2C11 and Gene Encoding the Same

Primers were designed based on the GmPP2C11 sequence information predicted by the phytozome website, and PCR amplification was conducted using the leaf cDNA of Nannong 1138-2 as a template.

Upstream primer GmPP2C11-F:
(SEQ ID NO: 9)
ACACCCGTGGTTTCACATTA;
Downstream primer GmPP2C11-R:
(SEQ ID NO: 10)
TCTCTTCCACCAGTTCAAACAT.

The GmPP2C11 gene was amplified from a total RNA of soybean leaf organs by reverse transcription PCR (RT-PCR). The soybean leaf tissue was crushed in a mortar, added into a 1.5 mL EP tube containing lysis solution, shaken thoroughly, and then transferred to a glass homogenizer. After homogenization, the mixture was transferred to a 1.5 mL EP tube, and total RNA was extracted using a plant total RNA extraction kit (TIANGEN DP404). The quality of total RNA was identified by formaldehyde denaturing gel electrophoresis, and the RNA was quantified by spectrophotometer. The total RNA was used as a template to allow reverse transcription according to the instructions of a reverse transcription kit provided by Takara Company to synthesize the first chain of cDNA. PCR amplification was conducted. The PCR system included: 2 μL of cDNA (0.05 μg), 2 μL each of upstream and downstream primers (10 μM), 25 μL of 2×PhantaMaxBuffer, 1 μL of dNTP (10 mM), and 1 U of Phanta Max Super-Fidelity DNA polymerase (Vazyme), and a balance of 50 μL ultrapure water. The PCR program on a Bio-RAD PTC200 PCR instrument included: initial denaturation at 94° C. for 3 min; a total of 35 cycles of denaturation at 94° C. for 15 s, annealing at 58° C. for 15 s, and extension at 72° C. for 45 s; extension at 72° C. for 5 min to terminate the reaction, and storage at 4° C. The PCR product was recovered and cloned into a pGEM-Teasy vector. After sequencing, the cDNA sequence of the soybean GmPP2C11 gene with a complete coding region, SEQ ID NO: 1, was obtained. The gene had a total length of 1,272 bp, and could encode the amino acid of SEQ ID NO: 2.

### Example 2

### Expression Profile of GmPP2C11 in Different Soybean Organs

RNA was extracted from roots, stems, leaves, flowers, pods and seeds of soybean cultivars Nannong 1138-2 and Kefeng 1, and reverse-transcribed into cDNA for RT-PCR.

Steps for the extraction of total RNA were the same as those in Example 1. A soybean constitutively expressed gene Tubulin was used as a reference gene, and its amplification primers included: Tubulin forward primer sequence: GGAGTTCACAGAGGCAGAG (SEQ ID NO: 11), and Tubulin reverse primer sequence: CACTTACGCATCACATAGCA (SEQ ID NO: 12). Real-time fluorescence quantitative PCR was conducted using the cDNA from different soybean tissues or organs as a template. The amplification primers of the GmPP2C11 gene included: GmPP2C11-qPCR-F: TGCTGATGGTGTAGGTGGCT (SEQ ID NO: 13), and GmPP2C11-qPCR-R: ATTTGCGTGAGCTTCCCTTA (SEQ ID NO: 14). The results (FIG. 2) showed that the expression of GmPP2C11 gene was relatively high in pods.

### Example 3

### Subcellular Localization of GmPP2C11 Gene

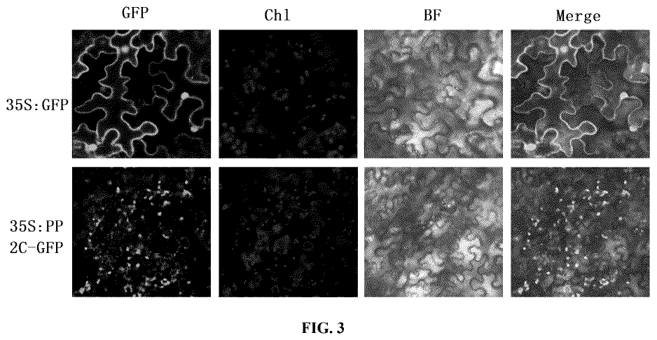

Subcellular localization was conducted using transient expression in Nicotiana benthamiana, with a vector pAN580 and primers including: GmPP2C11-F: acaaatctatctctctcgagATGCCTGGCATTCTCTCAAG (SEQ ID NO: 15), and GmPP2C11-R: gctcaccatggatccCTCACTCACTGAACCTGATATG (SEQ ID NO: 16). PCR amplification was conducted, and after the target band was correctly identified, gel extraction was performed. The gel-extracted product was ligated to a subcellular localization vector pAN580-GmPP2C11 (with the gene located at the N-terminal of GFP) by homologous recombination. Nicotiana benthamiana cells were cultured for 48 h after the expression and laser irradiation with a laser confocal microscope (Zeiss, LSM780) was completed to generate green fluorescent signals for protein localization. Images were observed and photographed. The results are shown in FIG. 3. The empty vector plasmid was distributed throughout the entire cell, while the GmPP2C11: GFP fusion protein was also distributed in the chloroplasts, where it merged with the chloroplast's red fluorescence. This suggests that the GmPP2C11 gene might function in chloroplasts.

### Example 4

### Use of GmPP2C11 Gene in Genetic Engineering

The coding sequence (CDS) of the GmPP2C11 gene was inputted into CRISPR-P (crispr.hzau.edu.cn/CRISPR2/) to obtain sgRNAs. The sgRNAs with high targeting efficiency and low off-target rate were selected and ligated to a PSCM vector driven by U3 and U6 promoters. In order to improve the targeting efficiency, 3 sgRNAs were selected:

GmPP2C11-sgF1:
(SEQ ID NO: 3)
TAATCTGACAAGAGTGTTAAgttttagagctagaaatagcaag,
GmPP2C11-sgR1:
(SEQ ID NO: 4)
TTAACACTCTTGTCAGATTAaatccatatgttttcctgggac;
GmPP2C11-sgF2:
(SEQ ID NO: 5)
TCCCATATCAGTTGGAGAGTgttttagagctagaaatagcaag,
GmPP2C11-sgR2:
(SEQ ID NO: 6)
ACTCTCCAACTGATATGGGAtgaccagacatgtcacgcttagt;
GmPP2C11-sgF3:
(SEQ ID NO: 7)
GTTGAACATGCAATTAGGGCgttttagagctagaaatagcaag,
GmPP2C11-sgR3:
(SEQ ID NO: 8)
GCCCTAATTGCATGTTCAACaatccatatgttttcctgggac.

They each were ligated into two PSCM vectors, and the two successfully-ligated PSCM vectors were dual-ligated to construct a tetra-cistronic vector with multiple sgRNAs to allow soybean target gene knockout in tissue culture experiments. The PSCM vector PSCM-GmPP2C11 was transformed into an Agrobacterium tumefaciens strain EHA105 using a freeze-thaw method.

The soybean seeds that showed no surface defect, had full grains, and exhibited a uniform seed coat color were selected and sterilized in a fume hood. Chlorine gas produced through the chemical reaction HCl (concentrated)+NaClO→Cl₂↑+NaOH (a volume ratio of concentrated hydrochloric acid to sodium hypochlorite was about 1:10) was used for sterilization. In the experiment, 120 mL of NaClO was placed in a conical flask. The soybeans in the culture dish were placed in a dryer, and the conical flask was placed in the center of the dryer and covered with a lid. 15 mL of concentrated HCl was slowly added from a top side of the dryer through a separatory funnel to allow sterilization for 7 h.

Seed germination: after sterilization, the seeds were fully blown with chlorine in a clean bench and vertically inserted into a pre-prepared and solidified SG4 germination solid medium such that the medium covered half of the hilum.

Inoculation: about 120 mL of a YEB broth with antibiotics Kan and Rif was added into a conical flask, 1.5 mL of a small amount of bacterial solution was added, and shaken at 28° C. and 200 rpm until OD₆₀₀=0.9.

Agrobacterium infection: the shaken bacterial solution was centrifuged at 5,000 rpm for 10 min at room temperature, a resulting supernatant was discarded, then co-culture medium (CCM) was added into two centrifuge tubes, suspended by shaking, adjusting an optical density (OD) to OD₆₀₀=0.6. Five days after the soybean seeds germinated, a part of the hypocotyl was removed to retain 5 mm to 10 mm, then the seeds were cut off along the cotyledons and hypocotyl, the true leaves were removed, and then several cuts were slightly made using a knife along the direction of the hypocotyl at the cotyledon node. Treated explants and the bacterial suspension obtained were poured into a sterilized jar, and co-cultivated for 30 min to 40 min at 28° C. and 120 rpm. Finally, the explants were taken out and placed on a filter paper on solid CCM with the cotyledonary node side facing downwards, where 14 explants were placed in each petri dish and cultivated for 5 d at 25° C. in the dark.

Induction of clustered buds: after 5 d of co-cultivation, the explants were sterilized with sterile water and Wash-Liquid, and excessively long hypocotyls were cut off to leave about 5 mm to 10 mm. The explants were inserted into solid selection and induction medium (SIM) without glufosinate at an angle of 45° with the growth point upward, where 8 explants were placed in each petri dish and cultivated for 15 d at 26° C. under light. After 15 d, the big buds and part of the hypocotyls were removed, and the explants with clustered buds were transferred to solid SIM supplemented with 6 mg/L glufosinate to allow screening culture for 15 d.

Elongation: cotyledons, dead leaves, and part of the hypocotyls of the explants that were not completely dead were removed, and the explants were transferred into solid shoot elongation medium (SEM) supplemented with 4 mg/L glufosinate to allow cultivation for 15 d. The dead leaves and part of the hypocotyls were removed while the explants were replaced on a new solid SEM at a cycle of 15 d, with the glufosinate concentration gradually decreasing.

Rooting: when the shoot of the explant grew to about 6 cm, the bottom of the explant was cut off, a cross-shaped incision was made at the base of the stem. The explants were transferred into a rooting medium (RM) for cultivation, and induced roots could be seen after about 10 d.

Hardening of seedlings: an appropriate amount of sterile water was poured into the bottle and the tissue culture seedlings were cultured at 26° C. for about 5 d and then transplanted. When the number and length of roots are appropriate, the tissue culture seedlings were separated from the medium, transferred into sterilized soil, and cultivated in an artificial incubator (16 h light/8 h dark, 25° C.).

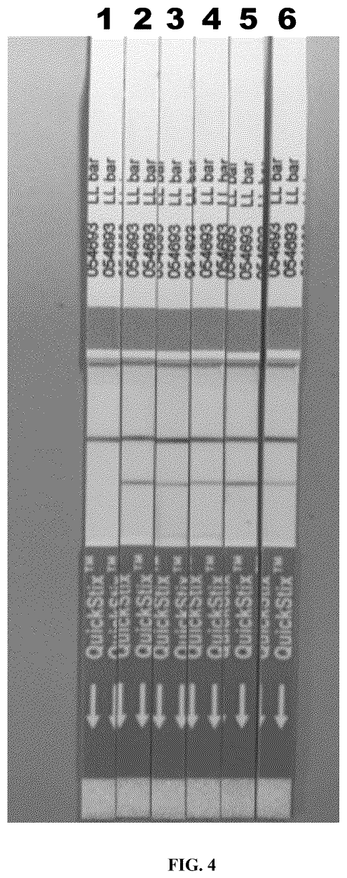

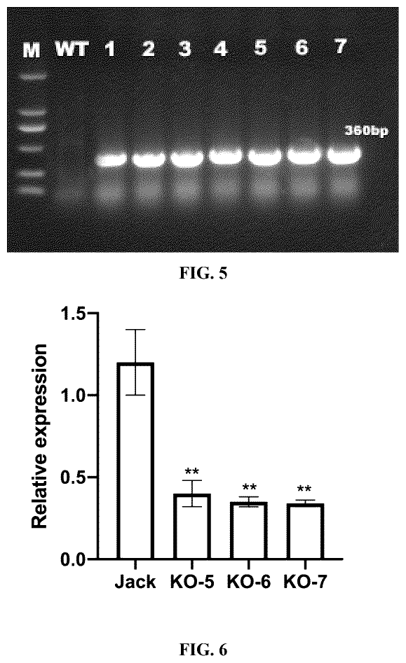

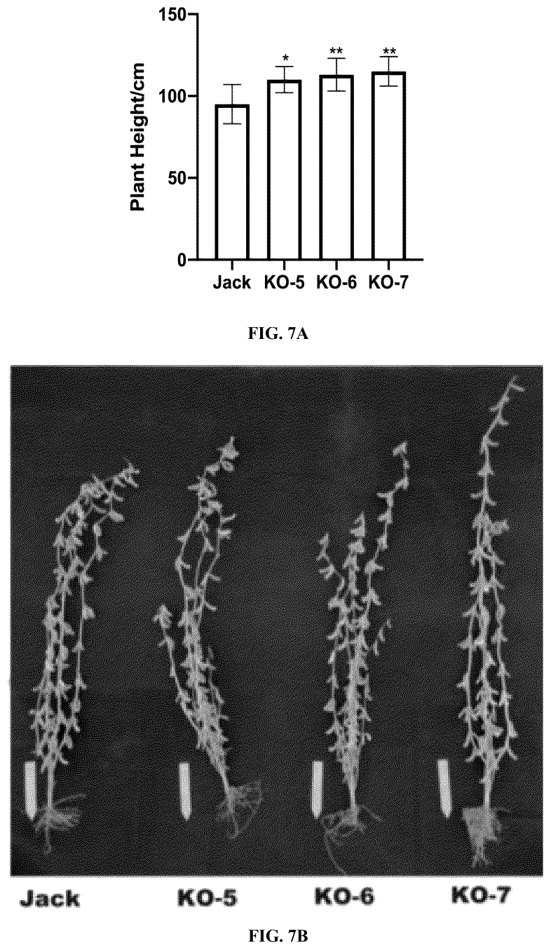

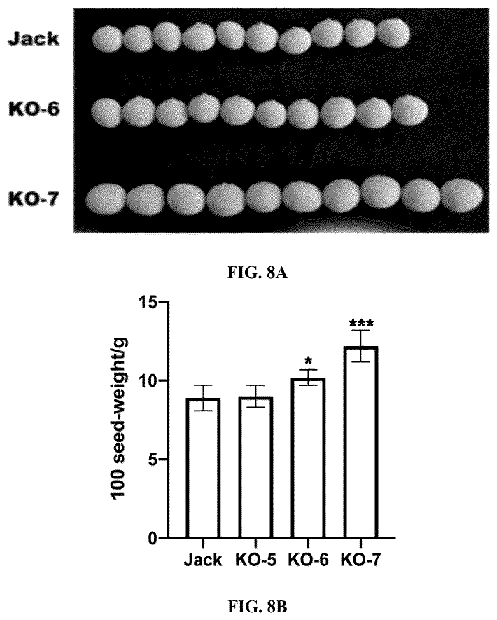

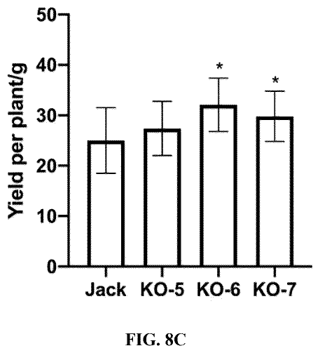

T₀ seedlings were tested with bar test strip and primers for the marker gene Bar, which were Bar-F: CGAGACAAGCACGGTCAACTT (SEQ ID NO: 17); and Bar-R: AAACCCACGTCATGCCAGTTC (SEQ ID NO: 18) (FIG. 4 and FIG. 5). Three homozygous edited lines were obtained from the indoor T₃ generation and GmPP2C11 expression analysis was conducted using GmPP2C11-qPCR-F: TGCTGATGGTGTAGGTGGCT (SEQ ID NO: 19), and GmPP2C11-qPCR-R: ATTTGCGTGAGCTTCCCTTA (SEQ ID NO: 20). The expression level of the GmPP2C11 gene in the three homozygous lines decreased significantly (FIG. 6). Compared with the receptor Jack, the three homozygous lines had a higher plant height at maturity, and their hundred-seed weight and yield per plant were significantly increased (FIG. 7A to FIG. 8C).

Although the above examples have described the present disclosure in detail, they are only a part of, not all of, the embodiments of the present disclosure. Other examples may also be obtained by persons based on the example without creative efforts, and all of these examples shall fall within the protection scope of the present disclosure.

1. A method for regulating a yield and/or a height of a plant, comprising subjecting the plant to genetic engineering with a soybean gene GmPP2C11 encoding a GmPP2C11 protein; wherein the soybean gene GmPP2C11 encoding the GmPP2C11 protein has the nucleotide sequence of SEQ ID NO: 1.

2. The method according to claim 1, wherein the regulation comprises: inhibiting expression of the soybean gene GmPP2C11 encoding the GmPP2C11 protein, thereby increasing plant height, a hundred-seed weight, and a yield per plant of soybean.

3. The method according to claim 1, wherein the genetic engineering comprises knock out of the soybean GmPP2C11 gene.

4. The method according to claim 3, wherein the knockout of the soybean GmPP2C11 gene specifically comprises: knocking out the soybean GmPP2C11 gene with a single guide RNA (sgRNA) knockout vector.

5. The method according to claim 4, wherein the sgRNA knockout vector is transformed into a plant cell or a plant tissue using a biological method, and a resulting transformed plant cell or a resulting transformed plant tissue is cultivated into a plant; and
the biological method is selected from the group consisting of a Ti plasmid method, an Ri plasmid method, a plant viral vector method, direct DNA transformation, microinjection, electroporation, and an Agrobacterium-mediated method.

6. The method according to claim 1, wherein the plant is selected from the group consisting of a monocotyledon and a dicotyledon;
the monocotyledon is selected from the group consisting of rice, wheat, and corn; and
the dicotyledon is selected from the group consisting of tobacco, Arabidopsis thaliana, soybean, rapeseed, cucumber, tomato, poplar, turf grass, and alfalfa.

7. A method for improving a plant height, a hundred-seed weight, and a yield per plant of soybean by genetic engineering, comprising transforming to the soybean an sgRNA knockout vector comprising a soybean gene GmPP2C11 encoding a GmPP2C11 protein; wherein the soybean gene GmPP2C11 encoding the GmPP2C11 protein has the nucleotide sequence of SEQ ID NO: 1.

8. The method according to claim 7, wherein the sgRNA knockout vector is a tetra-cistronic vector with multiple sgRNAs;
the tetra-cistronic vector with multiple sgRNAs comprises a tetra-cistronic vector with sgRNA1, a tetra-cistronic vector with sgRNA2, and a tetra-cistronic vector with sgRNA3;
sgRNA1 is ligated to two gene-editing vectors, and resulting two successfully-ligated gene-editing vectors are dual-ligated to obtain the tetra-cistronic vector with sgRNA1; wherein the sgRNA1 comprises GmPP2C11-sgF1 having the nucleotide sequence of SEQ ID NO: 3 and GmPP2C11-sgR1 having the nucleotide sequence of SEQ ID NO: 4;
sgRNA2 is ligated to two gene-editing vectors, and resulting two successfully-ligated gene-editing vectors are dual-ligated to obtain the tetra-cistronic vector with sgRNA2; wherein the sgRNA2 comprises GmPP2C11-sgF2 having the nucleotide sequence of SEQ ID NO: 5 and GmPP2C11-sgR2 having the nucleotide sequence of SEQ ID NO: 6; and
sgRNA3 is ligated to two gene-editing vectors, and resulting two successfully-ligated gene-editing vectors are dual-ligated to obtain the tetra-cistronic vector with sgRNA3; wherein the sgRNA3 comprises GmPP2C11-sgF3 having the nucleotide sequence of SEQ ID NO: 7 and GmPP2C11-sgR3 having the nucleotide sequence of SEQ ID NO: 8.

9. The method according to claim 8, wherein each of the gene-editing vectors is driven by a U3 promoter or a U6 promoter.

10. An sgRNA knockout vector for a soybean gene GmPP2C11 encoding a GmPP2C11 protein, wherein the sgRNA knockout vector knocks out the soybean GmPP2C11 gene.

11. The sgRNA knockout vector according to claim 10, wherein the sgRNA knockout vector is a tetra-cistronic vector with multiple sgRNAs;
the tetra-cistronic vector with multiple sgRNAs comprises a tetra-cistronic vector with sgRNA1, a tetra-cistronic vector with sgRNA2, and a tetra-cistronic vector with sgRNA3;
sgRNA1 is ligated to two gene-editing vectors, and resulting two successfully-ligated gene-editing vectors are dual-ligated to obtain the tetra-cistronic vector with sgRNA1; wherein the sgRNA1 comprises GmPP2C11-sgF1 having the nucleotide sequence of SEQ ID NO: 3 and GmPP2C11-sgR1 having the nucleotide sequence of SEQ ID NO: 4;
sgRNA2 is ligated to two gene-editing vectors, and resulting two successfully-ligated gene-editing vectors are dual-ligated to obtain the tetra-cistronic vector with sgRNA2; wherein the sgRNA2 comprises GmPP2C11-sgF2 having the nucleotide sequence of SEQ ID NO: 5 and GmPP2C11-sgR2 having the nucleotide sequence of SEQ ID NO: 6; and
sgRNA3 is ligated to two gene-editing vectors, and resulting two successfully-ligated gene-editing vectors are dual-ligated to obtain the tetra-cistronic vector with sgRNA3; wherein the sgRNA3 comprises GmPP2C11-sgF3 having the nucleotide sequence of SEQ ID NO: 7 and GmPP2C11-sgR3 having the nucleotide sequence of SEQ ID NO: 8.

12. The sgRNA knockout vector according to claim 10, wherein each of the gene-editing vectors is driven by a U3 promoter or a U6 promoter.
